(19) United States
(12) Patent Application Publication
(10) Pub. No.: US 2009/0012595 A1
(43) Pub. Date: Jan. 8, 2009

(54) Therapeutic Drug-Eluting Endoluminal Covering

(76) Inventors: Dror Seliktar, Haifa (IL); Rafael Beyar, Haifa (IL)

Correspondence Address: MARTIN D. MOYNIHAN d/b/a PRTSI, INC., P.O. BOX 16446, ARLINGTON, VA, 22215, US

(21) Appl. No.: 10/582,847

(22) PCT Filed: Dec. 15, 2004
(86) PCT No.: PCT/IL04/01129

§ 371 Date: Sep. 10, 2008

Related U.S. Application Data
(60) Provisional application No. 60/529,093, filed on Dec. 15, 2003.

(51) Int. Cl. A61F 2/82; A61K 38/18; A61F 2/92; A61F 2/84; C08L 89/00; C08L 1/00

(52) U.S. Cl. 623/1.11; 514/12; 623/1.46; 623/1.43; 623/1.2; 525/54.1; 525/54.21; 525/54.2

(57) ABSTRACT

The present invention is of methods of preventing restenosis and promoting vascular re-healing. Specifically, the present invention is of a method of exposing the luminal wall of a blood vessel to a substance by deploying a drug-eluting polymer film inside the lumen of a blood vessel during or following angioplasty.

### FIELD AND BACKGROUND OF THE INVENTION

The present invention relates to compositions and methods for exposing a luminal wall of a biological vessel to a substance. Specifically, the compositions and methods of the present invention can be used to prevent and/or treat restenosis following angioplasty.

Atherosclerosis affects 20% of the population and remains the main cause of death in the Western world. Atherosclerosis is a progressive disease manifested by a restricted blood flow leading to a progressive dysfunction of the arteries, tissues or organs downstream of the site of blockage. Thus, atherosclerosis may be associated with myocardial infraction, heart attacks, infraction in the brain, infarctions in the lower extremities, and subsequently cerebrovascular incidents, strokes, and/or organ amputations.

Treatment of atherosclerosis includes bypass grafting of venous, percutaneous coronary intervention (PCI, i.e., balloon angioplasty with or without stent placement), atherectomy and most recently, in cardiac perfusion and laser transmyocardial revascularization.

PCI represents an attractive alternative to surgical revascularization and has become the most accepted treatment, worldwide, to coronary stenosis. The combination of metallic stents and balloon angioplasty has significantly improved the efficacy of PCI. It is estimated that almost 80% of contemporary procedures use coronary stents. However, in 15-50% of the cases, 6 to 9 months following balloon and/or stent placement, restenosis occurs. Restenosis is a process of re-narrowing the blood vessel as a result of advanced de-endothelialization and/or vascular expansion which leads to the migration of smooth muscle cells (SMC) and the deposition of extracellular matrix (ECM) at the site of angioplasty or stent placement.

To overcome such limitations, new approaches utilizing various stent designs have been developed. Stents have been made from various types of metals and polymers and in various shapes. It was found that tubular and corrugated stents are more efficient in preventing restenosis than coiled or meshwired stents; likewise, stents with thin struts are advantageous over stents with thick-strut. On the other hand, gold, phosphorylcholine or heparin-coated stents did not present an advantage over bare, stainless-steel stents (Lau K W et al., 2004; J. Invasive Cardiol. 16: 411-6).

Further developments in the field of stent coating included drug-eluting stents. Stents were designed to elute specific drugs such as antiproliferative agents capable of slowing down the SMC response to the injury caused by balloon angioplasty and/or stent placement. Such drug-eluting stents caused a significant reduction in acute re-occlusion and neointimal hyperplasia, the major causes of in-stent restenosis. However, in several cases, especially in peripheral vessels such as infrarenal aorta, pelvic and lower extremity vasculature, the effect of drug-eluting stents is limited due to the large surface area needing treatment. In such cases, most of the injury site is left uncovered by the drug-eluting stent struts. In fact, coated stents typically cover less than 10 percent of the peripheral vessel injury site. In addition, the high concentration of the drug needed for adequate delivery to such a large surface area often results in exposing the region at the interface between the stent and the artery wall to high drug concentrations and to further adverse effects. Thus, despite the widespread acceptance of stent coatings, this strategy exhibits limited long-term clinical efficacy in vascular healing.

In order to overcome the inherent limitations of stenting in non-coronary vessels, a novel approach named endoluminal paving was proposed nearly a decade ago by Slepian et al (Slepian, M J, Cardiol Clin. 1994, 12: 715-37; Slepian, M J, Semin Interv Cardiol. 1996, 1: 103-16). This approach uses a biodegradable hydrogel which covers the entire balloon injury site immediately following balloon inflation and combines the benefits of local anti-thrombotic blood barrier preventing thrombosis with the conventional drug delivery paradigm for treating intimal hyperplasia. The primary advantage of endoluminal paving over conventional drug-eluting stents is the ability to uniformly deliver drugs to the entire vessel injury. However, the major limitation of such an approach is the technical hurdle of paving the artery with an adherent, microns-thick, hydrophilic polymeric hydrogel biomaterial, which easily binds to the distending tissue surface. To improve the physical characteristics of hydrogel biomaterial, various cross-linking modifications have been employed. However, the increase in hydrogel stiffness resulted in brittle materials which were more susceptible to failure under cyclic hemodynamic loading. Thus, despite the comparatively impressive preliminary results in animals (Hill-West J L, et al., 1994, Proc. Natl. Acad. Sci. USA. 91: 5967-71), this approach resulted in no published clinical studies.

There is thus a widely recognized need for, and it would be highly advantageous to have, a method of preventing restenosis and promoting vascular re-healing devoid of the above limitations.

### SUMMARY OF THE INVENTION

According to one aspect of the present invention there is provided a method of exposing a luminal wall of a biological vessel to a substance, comprising: (a) inserting a rolled polymer film including the substance into a lumen of the biological vessel; and (b) unrolling the rolled polymer film in the lumen of the biological vessel thereby exposing the luminal wall of the biological vessel to the substance.

According to another aspect of the present invention there is provided a method of preventing restenosis in an individual in need thereof, comprising: (a) inserting a rolled polymer film including a substance into a lumen of a blood vessel of the individual; and (b) unrolling the rolled polymer film in the lumen of the blood vessel thereby exposing the luminal wall of the blood vessel to the substance and preventing restenosis in the individual.

According to yet another aspect of the present invention there is provided a method of promoting vascular re-healing in an individual in need of an angioplasty procedure, comprising: (a) inserting a rolled polymer film including a substance capable of promoting vascular re-healing into a lumen of a blood vessel of the individual; and (b) unrolling the rolled polymer film in the lumen of the blood vessel thereby exposing the luminal wall of the blood vessel to the substance and promoting vascular re-healing in the individual in need of the angioplasty procedure.

According to still another aspect of the present invention there is provided a composition-of-matter comprising polyethylene glycol (PEG) attached to alginate.

According to an additional aspect of the present invention there is provided a polymer film comprising polyethylene glycol (PEG) attached to alginate.

According to yet an additional aspect of the present invention there is provided a drug-eluting film comprising polyethylene glycol (PEG) attached to alginate and at least one drug

According to still an additional aspect of the present invention there is provided a method of preventing thrombosis at a luminal wall of a blood vessel, comprising: (a) inserting a rolled polymer film into a lumen of the blood vessel; and (b) unrolling the rolled polymer film in the lumen of the blood vessel thereby preventing thrombosis at the luminal wall of the blood vessel.

According to further features in preferred embodiments of the invention described below, the rolled polymer film is rolled over a stent.

According to still further features in the described preferred embodiments the stent is positioned over a balloon catheter used in angioplasty.

According to still further features in the described preferred embodiments inserting the rolled polymer is effected using a catheter.

According to still further features in the described preferred embodiments unrolling the rolled polymer is effected using the balloon catheter used in angioplasty.

According to still further features in the described preferred embodiments unrolling the rolled polymer is effected using a self-expandable stent.

According to still further features in the described preferred embodiments the polymer film is biodegradable.

According to still further features in the described preferred embodiments the substance forms a part of the polymer film.

According to still further features in the described preferred embodiments the substance coats the polymer film.

According to still further features in the described preferred embodiments the substance included in the polymer film is selected from the group consisting of PEG-alginate, alginate, PEG-fibrinogen, PEG-collagen, PEG-albumin, collagen, fibrin, and alginate-fibrin.

According to still further features in the described preferred embodiments the PEG constitute of the PEG-alginate is selected from the group consisting of PEG-acrylate (PEG-Ac) and PEG-vinylsulfone (PEG-VS).

According to still further features in the described preferred embodiments the PEG-Ac is selected from the group consisting of PEG-DA, 4-arm star PEG multi-Acrylate and 8-arm star PEG multi-Acrylate.

According to still further features in the described preferred embodiments the PEG-DA is a 4-kDa PEG-DA, 6-kDa PEG-DA, 10-kDa PEG-DA and/or 20-kDa PEG-DA.

According to still further features in the described preferred embodiments a weight ratio between the 4-kDa PEG-DA to the alginate is 0.1 gram to 1.0 gram, respectively.

According to still further features in the described preferred embodiments the alginate is sodium alginate.

According to still further features in the described preferred embodiments the substance included in the polymer film is a drug.

According to still further features in the described preferred embodiments the drug is selected from the group consisting of an antiproliferative drug, a growth factor, a cytokine, and an immunosuppressant drug.

According to still further features in the described preferred embodiments the antiproliferative drug is selected from the group consisting of rapamycin, paclitaxel, tranilast, and trapidil.

According to still further features in the described preferred embodiments the growth factor is selected from the group consisting of Vascular Endothelial Growth Factor (VEGF), and angiopeptin.

According to still further features in the described preferred embodiments the cytokine is selected from the group consisting of M-CSF, IL-1beta, IL-8, beta-thromboglobulin, EMAP-II, G-CSF, and IL-10.

According to still further features in the described preferred embodiments the immunosuppressant drug is selected from the group consisting of sirolimus, tacrolimus, and Cyclosporine.

According to still further features in the described preferred embodiments the substance is a non-thrombogenic and/or an anti-adhesive substance.

According to still further features in the described preferred embodiments the non-thrombogenic and/or an anti-adhesive substance is selected from the group consisting of tissue plasminogen activator, reteplase, TNK-tPA, a glycoprotein IIb/IIIa inhibitor, clopidogrel, aspirin, heparin, enoxiparin and dalteparin.

According to still further features in the described preferred embodiments the biological vessel is selected from the group consisting of a blood vessel, an air tract vessel, a urinary tract vessel, and a digestive tract vessel.

According to still further features in the described preferred embodiments the blood vessel is selected from the group consisting of an artery and a vein.

According to still further features in the described preferred embodiments the individual suffers from a disease selected from the group consisting of atherosclerosis, diabetes, heart disease, vacular disease, peripheral vascular disease, coronary heart disease, unstable angina and non-Q-wave myocardial infarction, and Q-wave myocardial infarction.

The present invention successfully addresses the shortcomings of the presently known configurations by providing a method of exposing the luminal wall of a biological vessel to a substance.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1a-b are schematic illustrations depicting the process of coating a balloon catheter with a drug-eluting sheet. FIG. 1a—illustrates the rolling of a thin, biodegradable drug-eluting sheet overtop of a balloon catheter containing a metallic stent; FIG. 1b—illustrates the completely rolled sheet over the catheter. Noteworthy that once the sheet is completely rolled over the catheter it is secured in place with a very mild medical grade biological adhesive.

FIG. 2 is a schematic illustration of a cross section of micron-thin, biodegradable, drug-containing, biodegradable sheet rolled over a balloon catheter holding a metallic stent. Shown are the catheter lumen which is proceeded by the wall of the catheter (arrow 1), the un-inflated lumen of the balloon (arrow 2), the wall of the balloon (arrow 3), the stent struts (arrow 4), and the rolled, drug-eluting sheet (arrow 5).

FIGS. 3a-b are schematic illustrations depicting the unrolling of the drug-eluted sheet onto the artery wall. A balloon catheter with a metallic stent and a drug eluting sheet rolled overtop is inflated inside the vessel lumen (FIG. 3a), causing the stent to expand and the drug eluting sheet to unroll onto the artery wall (FIG. 3b). Following the procedure, the expanded stent fixes the unrolled drug-eluting sheet on the vessel wall and the vessel lumen is expanded (FIG. 3c).

FIGS. 4a-d are schematic illustrations depicting the deployment of the polymer film of the present invention into an atherosclerotic artery. A pre-cast, microns-thick alginate-PEG film is cut to the exact dimensions of the stent length, following which the film is pre-wetted for 5 minutes before being wrapped around the outer wall of the stent struts (FIG. 4a). The film is wrapped around the stent and is secured in place by applying a thin strip of mild fibrin sealant on the outer edge of the film and securing the edge to the opposing side on the wrapped film (FIG. 4b). Finally, the secured film, stent, and balloon catheter are inserted into the atherosclerotic region of the artery wall for stent and film deployment (FIG. 4c). During stent and film deployment, the fibrin sealant on the edge of the film is sheared, causing the release and unraveling of the polymer film with the expansion of the balloon and stent struts (FIG. 4d).

FIGS. 5a-b are graphs depicting the uniaxial tensile mechanical properties of dry (FIG. 5a) and wet (FIG. 5b) Alginate, PEG or PEG-Alginate films. Dry and wet films were strained using an Instron single column testing apparatus under constant strain loading as the tensile stress is measured. Note the significantly higher tensile stress of dry films (FIG. 5a) as compared with that of wet films (FIG. 5b). Also note the alginate films were significantly stiffer than the PEG-alginate films (FIGS. 5a-b), demonstrating that the alginate constitute dominates the material stiffness and strength. The combination of PEG-alginate with or without UV photoinitiation has a significant effect on the stiffness of the material; the PEG acts as a plasticizing agent which reduces the material modulus. The PEG-alginate films are also less brittle than the alginate film.

FIGS. 6a-b are graphs depicting the dependency of cross-linking of the alginate films (FIG. 6a) or the PEG-alginate film (FIG. 6b) on the concentration of CaCl₂ cross-linker. The swelling ratio (SR) immediately after cross-linking is used to assess the degree of cross-linking; smaller swelling ratio indicates higher cross-linking. Note the minimal swelling (and highest cross-linking) of the alginate films in the presence of 15% (w/v) of CaCl₂ (FIG. 6a). Also note that the addition of PEG to the alginate network does not significantly affect the cross-linking properties of the alginate-based films (FIG. 6b).

FIGS. 7a-c are scanning electron micrographs of PEG (FIG. 7a), alginate (ALG, FIG. 7b) or PEG-alginate (PEG-ALG; FIG. 7c) films. Note the highly dense and smooth surface present in the alginate film (FIG. 7b) as compared with the PEG film (FIG. 7a). Also note that the addition of PEG to the alginate network only slightly affects the surface characteristics of the PEG-alginate films (FIG. 7c).

FIG. 8 is a graph depicting the release of PEG from the alginate-based films. PEG release is measured by quantifying the PEG remaining in the PEG-alginate films using an iodine assay. Note that the amount of PEG present in the alginate network is initially higher in UV cross-linked alginate sheets. However, after 50 hours, the amounts of PEG remaining in the UV cross-linked (UV+) and control (UV−) films is nearly identical, demonstrating that the release of PEG from the alginate-based film is independent of UV photoinitiation. In both cases, the amount of PEG remaining in the PEG-alginate films after 21 days is approximately 35% of the original amount on day zero.

FIGS. 9a-b are graphs depicting the dependency of the degradation of alginate-based films on the ionic concentration of the suspension buffer. Degradation of the films is measured by mechanical testing using an Instron single column testing apparatus under uniaxial constant strain loading, which measures the modulus (E) of the material. The degradation parameter is obtained by normalizing the modulus of partially deteriorated films with those of intact films suspended in deionized water. Note that the degradation of the alginate-based films is highly responsive to the concentration of PBS buffer used in the experiment. After an initial drop in stiffness, the films do not undergo additional degradation in their respective buffer solutions (FIG. 9a). In contrast, when the buffer solution is replenished during each time interval, the degradation of the alginate-based films in significantly affected (FIG. 9b). The alginate films exhibit rapid deterioration, depending on the ionic strength of the suspension buffer, to the point that they can no longer be characterized.

FIGS. 10a-b are graphs depicting the kinetics of Paclitaxel release from endoluminal films in H₂O (FIG. 10a) or PBS (FIG. 10b). Paclitaxil release was measured using the UV/VIS spectrophotometer at an absorbance wavelength of 232 nm. A=alginate; A+P=PEG-Alginate; UV (+) or (−)=the presence or absence, respectively, of UV cross-linking of the PEG constitute of the polymer films. Note that the release of the paclitaxel drug from the alginate films is similar to that of the PEG-alginate films (FIGS. 10a-b). In addition, UV cross-linked films containing PEG (UV+) do not appear to release the PEG slower than their corresponding negative controls (UV−). Likewise, the percent drug loaded into the films (5% vs. 10%, v/v) does not appear to have a significant impact on the release of the drug (FIGS. 10a-b). On the other hand, note that the release of drug from the polymer film into water (H₂O; FIG. 10a) was significantly slower than in the presence of phosphate buffer saline (PBS) (FIG. 10b).

### DESCRIPTION OF THE PREFERRED EMBODIMENTS

The present invention is of compositions and methods for exposing a luminal wall of a biological vessel to a substance. Specifically, the compositions and methods of the present invention can be used to prevent and/or treat restenosis following angioplasty.

The principles and operation of the method of exposing the luminal wall of a blood vessel with a substance according to the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Atherosclerosis affects 20% of the population and remains the main cause of death in the Western world. The most attractive and common approach for treating atherosclerosis is based on percutaneous coronary intervention (PCI), i.e., balloon angioplasty with or without stent placement.

However, one of the major complications in PCI is the development of restenosis, which occurs in 15-50% of the cases approximately 6 to 9 months following balloon and/or stent placement. Restenosis results from de-endothelialization and smooth muscle cell (SMC) injury which leads to SMC activation and deposition of extracellular matrix (ECM) at the site of angioplasty or stent placement.

Several approaches have been developed to prevent restenosis. These include design of stents with various shapes, dimensions and/or materials [Lau, 2004 (Supra)]. Additionally, drug-eluting stents were developed with various antiproliferative drugs such as rapamycin, paclitaxel, tranilast, and trapidil. However, in several cases, and especially in peripheral vessels such as infrarenal aorta, pelvic and lower extremity vasculature, the effect of drug-eluting stents is limited by the large surface area needing treatment. In such cases, most of the injury site is left uncovered by the drug-eluting stent struts. In fact, coated stents typically cover less than 10 percent of the peripheral vessel injury site. In addition, the high concentration of the drug needed for adequate delivery to such a large surface area often results in exposing the region at the interface between the stent and the artery wall to high drug concentrations which can lead to adverse effects.

Another approach for preventing restenosis involves endoluminal paving and uses a biodegradable hydrogel to cover the entire balloon injury site immediately after balloon inflation [See Slepian, 1994 (Supra); Slepian, 1996 (Supra)]. However, paving the artery with an adherent, microns-thick polymeric hydrogel biomaterial is technically difficult and practically unachievable. In addition, the prior art polymers used for endoluminal paving exhibit inherent properties of swelling and deformation and are therefore unsuitable for endoluminal paving. Thus, despite the comparatively impressive preliminary results in animals [Hill-West, 1994 (Supra)] this approach resulted in no published clinical studies.

While reducing the present invention to practice, the present inventors have generated a novel biodegradable polymer film which can be placed within the lumen of a blood vessel and function to promote vascular re-healing and prevent restenosis. In addition, the present inventors have uncovered a new composition-of-matter including polyethylene glycol (PEG) and alginate which has unique inherent properties that are highly suitable for using in promoting vascular re-healing and preventing restenosis.

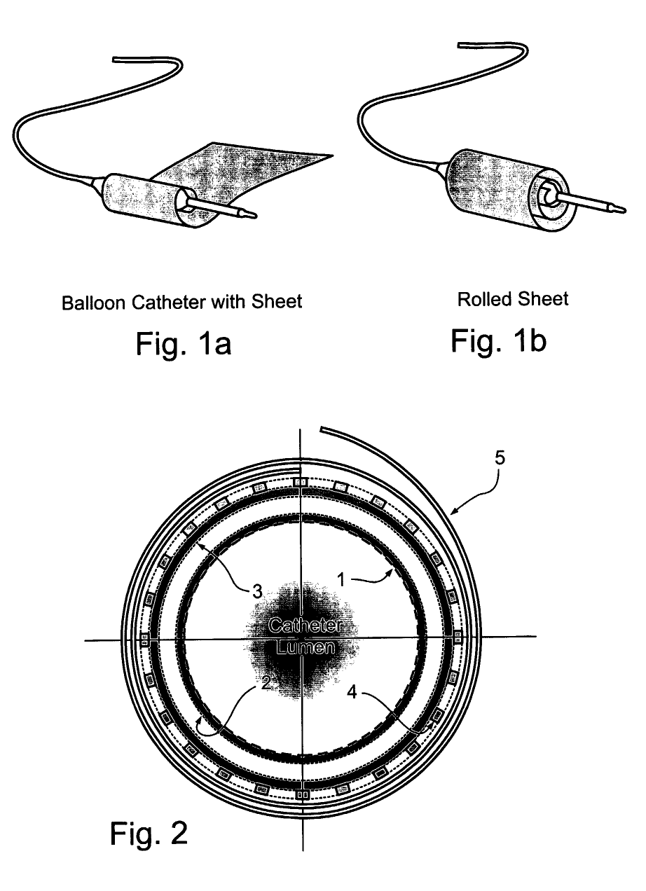

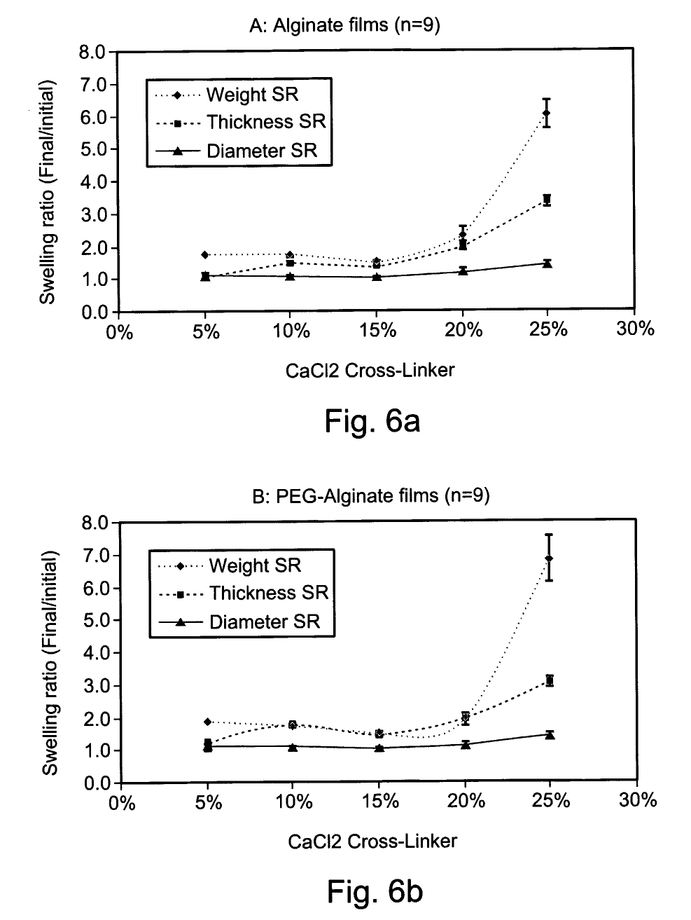

As is shown in FIGS. 1-4 and described in Example 1 of the Examples section which follows the polymer film of the present invention is rolled around a stent strut which is positioned over a balloon catheter used for angioplasty. Following the insertion of the balloon catheter into the lumen of the blood vessel and its inflation, the stent is deployed, causing the polymer film to unroll against the luminal wall of the blood vessel. In addition, as is shown in Table 2, FIGS. 6a-b and described in Example 2 of the Examples section which follows, the PEG-alginate polymer of the present invention has unique swelling properties which are superior to those of prior art polymers and which make it highly suitable for endoluminal use. The PEG-alginate polymer of the present invention does not swell radially in an aqueous environment and as such is unlikely to delaminate or separate from the luminal interface of the blood vessel wall. Moreover, as is further described in Examples 2 and 3 of the Examples section which follows, the PEG-alginate polymer film of the present invention was capable of releasing Paclitaxel into the lumen of a rabbit abdominal aortic tissue using an in vitro organ culture system.

Thus, according to one aspect of the present invention there is provided a method of exposing a luminal wall of a biological vessel, such as a blood vessel, to a substance.

As used herein the phrase “exposing a luminal wall . . . to a substance” refers to making the luminal wall accessible to the substance of the present invention.

The phrase “luminal wall” as used here refers to the interior part of the biological vessel of the present invention through which the body fluid is contained, conveyed and/or circulated.

The phrase “biological vessel” as used herein refers to any tube, canal, and/or cavity in an organism, preferably a mammal, more preferably, a human being, in which a body fluid is contained, conveyed and/or circulated. Non-limiting examples of biological vessels which can be treated by the present invention include a blood vessel (e.g., aorta, right coronary artery, left circumflex artery, infrarenal aorta, pelvic and lower extremity vasculature), an air tract vessel (e.g., a trachea), a urinary tract vessel (e.g., urethra, kidney), a digestive tract vessel (e.g., an intestine, a stomach) and the like.

The method is effected by inserting a rolled polymer film including the substance into a lumen of the biological vessel; and unrolling the rolled polymer film in the lumen of the biological vessel thereby exposing the luminal wall of the biological vessel to the substance.

The polymer used by the present invention can be a synthetic polymer (i.e., a polymer made of a non-natural, non-cellular material), a biological polymer (i.e., a polymer made of cellular or acellular materials) and/or a polymer made of a hybrid material (i.e., composed of biological and synthetic materials).

Non-limiting examples of synthetic polymers which can be used along with the present invention include polyethylene glycol (PEG) (average Mw. 200; P3015, SIGMA), Hydroxyapatite/polycaprolactone (HA/PLC) [Choi, D., et al., 2004, Materials Research Bulletin, 39: 417-432; Azevedo M C, et al., 2003, J. Mater Sci. Mater. Med. 14(2): 103-7], polyglycolic acid (PGA) [Nakamura T, et al., 2004, Brain Res. 1027(1-2): 18-29], Poly-L-lactic acid (PLLA) [Ma Z, et al., 2005, Biomaterials. 26(11): 1253-9], Polymethyl methacrylate (PMMA) [average Mw 93,000, Aldrich Cat. # 370037; Li C, et al., 2004, J. Mater. Sci. Mater. Med. 15(1): 85-9], polyhydroxyalkanoate (PHA) [Zinn M, et al., 2001, Adv. Drug Deliv. Rev. 53(1): 5-21; Sudesh K., 2004, Med. J. Malaysia. 59 Suppl B: 55-6], poly-4-hydroxybutyrate (P4HB) [Dvorin E L et al., 2003, Tissue Eng. 9(3): 487-93], polypropylene fumarate (PPF) [Dean D, et al., 2003, Tissue Eng. 9(3): 495-504; He S, et al., 2000, Biomaterials, 21(23): 2389-94], polyethylene glycol-dimethacrylate (PEG-DMA) [Oral E and Peppas NA J, 2004, Biomed. Mater. Res. 68A(3): 439-47], beta-tricalcium phosphate (beta-TCP) [Dong J, et al., 2002, Biomaterials, 23(23): 4493-502], and nonbiodegradable polytetrafluoroethylene (PTFE) [Jernigan T W, et al., 2004. Ann. Surg. 239(5): 733-8; discussion 738-40].

Non-limiting examples of biological polymers which can be used along with the present invention include collagen, fibrin (Herrick S., et al., 1999, Int. J. Biochem. Cell Biol. 31: 741-6; Werb Z, 1997, Cell, 91: 439-42), alginate (Yang J et al., 2002, Biomaterials 23: 471-9), hyaluronic acid (Lisignoli G et al., 2002, Biomaterials, 2002, 23: 1043-51), gelatin (Zhang Y., et al., 2004; J Biomed Mater Res. 2004 Sep. 22; Epub ahead of print), and bacterial cellulose (BC) (Svensson A et al., 2005, Biomaterials, 6: 419-31).

Non-limiting examples of polymers made of hybrid materials which can be used along with the present invention include synthetic PEG which was cross-linked with short oligopeptides [Lutolf et al (2003) Biomacromolecules, 4: 713-22; Gobin and West (2002) Faseb J. 16: 751-3; Seliktar et al., (2004) J. Biomed. Mater. Res. 68A(4): 704-16; Zisch A H, et al, 2003; FASEB J. 17: 2260-2] or a hybrid polymer composed of a protein backbone and PEG cross-links [Almany and Seliktar (2005) Biomaterials May, 26(15):2467-77].

Preferably, the polymer film used by the present invention is biodegradable, i.e., capable of being degraded (i.e., broken down) in a physiological aqueous environment and is therefore made of biological material and/or a hybrid materials. Examples for such polymer films include, but are not limited to, PEG-alginate, alginate, collagen, fibrin, hyaluronic acid, gelatin, and bacterial cellulose (BC).

The dimensions of the polymer film of the present invention (length, width and thickness) are selected according to the biological vessel targeted for treatment. Typically, the polymer film is microns-thin and capable of being rolled and placed into a biological vessel.

For example, a polymer film which can be used to expose the endoluminal wall of the trachea to the substance of the present invention would have a width in a range of 40-50 mm, a length in a range of 10-150 mm and a thickness in the range of 10-300 μm. Preferably, for endoluminal covering of the trachea the polymer film of the present invention exhibits a width of 47 mm, a length of 100 mm and a width of 200 μm.

Similarly, a polymer film which can be used to expose the endoluminal wall of the duodenum of the stomach to the substance of the present invention would have a width in a range of 90-160 mm, a length in a range of 10-150 mm and a thickness in the range of 10-300 μm. Preferably, for endoluminal covering of the stomach the polymer film of the present invention exhibits a width of 120 mm, a length of 150 mm and a width of 200 μm.

Preferably, a polymer film which can be used to expose the endoluminal wall of the aorta to the substance of the present invention would have a width in a range of 70-85 mm, a length in a range of 30-150 mm and a thickness in the range of 10-300 μm. Preferably, for endoluminal covering of the aorta the polymer film of the present invention exhibits a width of 78 mm, a length of 100 mm and a width of 200 μm.

As is mentioned before, the rolled polymer film of the present invention includes a substance.

As used herein, the phrase “substance” refers to any physical material or matter with a particular or definite chemical constitution (e.g., a drug molecule or an agent with a therapeutic property). Preferably, the substance used by the present invention is used to form the polymer film (i.e., a synthetic or biological material used to make the polymer film as described hereinabove), or is coated thereupon or integrated therewithin (impregnated).

Preferably, the substance used by the present invention is a drug molecule or an agent having a therapeutic property such as an antiproliferative agent, a growth factor, and/or an immunosuppressant drug. Additionally or alternatively, the substance used by the present invention is a non-thrombogenic and/or an anti-adhesive molecule capable of preventing the absorption of proteins and/or coagulation factors to the polymer film of the present invention.

Non-limiting examples of antiproliferative drugs which can be used by the present invention include rapamycin (Pedersen S S et al., 2004; J Am Coll Cardiol. 44(5): 997-1001), paclitaxel (Lee C H et al., 2004; Heart. 90(12):1482), tranilast (Ishiwata S et al., J Am Coll Cardiol. 2000 April; 35(5):1331-7), Atorvastatin (Scheller B., et al., 2003; Z. Kardiol. 92(12):1025-8) and trapidil (Galassi A R, et al., 1999; Catheter Cardiovasc Interv. 46(2): 162-8).

Non-limiting examples of growth factors which can be used by the present invention include Vascular Endothelial Growth Factor (VEGF; Swanson N., et al., 2003; J. Invasive Cardiol. 15(12): 688-92), and angiopeptin (Armstrong J, et al., 2002; J. Invasive Cardiol. 14(5): 230-8).

Non-limiting examples for cytokines which can be used by the present invention include M-CSF, IL-1 beta, IL-8, beta-thromboglobulin, and EMAP-II (Nuhrenberg T G et al., 2004, FASEB J. November 16; (Epub ahead of print)], granulocyte-colony stimulating factor (GGSF) (Kong D, et al., Circulation. 2004 Oct. 5; 110(14):2039-46), and IL-10 (Mazighi M et al., Am J Physiol Heart Circ Physiol. 2004 August; 287(2):H866-71).

Non-limiting examples of immunosuppressants which can be used by the present invention include sirolimus (Saia F et al., 2004; Heart. 90(10): 1183-8), tacrolimus (Grube E, Buellesfeld L. Herz. 2004 March; 29(2):162-6), and Cyclosporine (Arruda J A et al., 2003, Am. J. Cardiol. 91: 1363-5).

Examples of suitable non-thrombogenic and/or anti-adhesive substances include, but are not limited to, tissue plasminogen activator, reteplase, TNK-tPA, glycoprotein IIb/IIIa inhibitors (e.g., abciximab, eptifibatide, tirofiban), clopidogrel, aspirin, heparin and low molecular weight heparins such as enoxiparin and dalteparin (Reviewed in Buerke M and Rupprecht H J, 2000. EXS 89:193-209).

According to presently preferred configurations, the polymer film of the present invention is made of a combination of PEG and alginate (PEG-alginate).

As is illustrated by the examples section which follows, the PEG-alginate polymer film of the present invention is prepared using a novel approach which enables the formation of a polymer film, which can be subjected to hydration without radial swelling and being highly flexible but exhibiting high tensile strength, and yet is biodegradable.

The PEG molecule used by the present invention to generate the PEG-alginate polymer can be linearized or branched (i.e., 2-arm, 4-arm, and 8-arm PEG) and at any molecular weight, e.g., 4 kDa, 6 kDa and 20 kDa for linearized or 2-arm PEG, 14 kDa and 20 kDa for 4-arm PEG, and 14 kDa and 20 kDa for 8-arm PEG and combination thereof.

As is described in Example 2 of the Examples section which follows the OH-termini of the PEG molecule can be reacted with a chemical group such as acrylate (Ac) which turns the PEG molecule into a functionalized PEG, i.e., PEG-Ac or PEG-vinylsulfone (VS). It will be appreciated that such chemical groups can be attached to linearized, 2-arm, 4-arm, or 8-arm PEG-OH molecules. Preferably, the PEG-Ac used by the present invention is PEG-DA, 4-arm star PEG multi-Acrylate and/or 8-arm star PEG multi-Acrylate.

Methods of preparing functionalized PEG molecules are known in the arts and are further described in Example 2 of the Examples section which follows.

The alginate component of the PEG-alginate polymer of the present invention can be any alginate known in the art, including, but not limited to, sodium alginate (Tajima S et al., Dent Mater J. 2004; 23(3):329-34), calcium alginate (Lee J S et al., 2004; J. Agric. Food Chem. 52: 7300-5), and glyceryl alginate (Int J. Toxicol. 2004; 23 Suppl 2:55-94), Preferably, the alginate component used to prepare the PEG-alginate of the present invention is sodium alginate.

Thus, the PEG-alginate polymer of the present invention is preferably prepared by mixing a precursor solution of alginate with functionalized PEG (e.g., PEG-DA).

It will be appreciated that the PEG and alginate components can be mixed at various weight or molar ratios.

Preferably, the weight ratio between PEG-DA (4-kDa) to alginate is at least 0.4 gram (PEG-DA) to 1.0 gram (alginate), more preferably, the weight ratio is 0.2 gram (PEG-DA) to 1.0 gram (alginate), most preferably, 0.1 gram (PEG-DA) to 1.0 gram (alginate).

It will be appreciated that in order to obtain a polymer, the PEG and alginate precursor molecules are preferably subjected to a cross-linking reaction.

Cross-linking of the polymer film of the present invention can be performed using methods known in the arts, including, but not limited to, cross-linking via photoinitiation (in the presence of an appropriate light, e.g., 365 nm), chemical cross-linking [in the presence of a free-radical donor] and/or heating [at the appropriate temperatures].

Preferably, cross-linking of the PEG constitute of the PEG-alginate polymer of the present invention is performed by subjecting the polymer precursor molecules to a free-radical polymerization reaction using photoinitiation.

Photoinitiation can take place using a photoinitiation agent (i.e., photoinitiator) such as bis(2,4,6-trimethylbenzoyl) phenylphosphine oxide (BAPO) (Fisher J P et al., 2001; J. Biomater. Sci. Polym. Ed. 12: 673-87), 2,2-dimethoxy-2-phenylacetophenone (DMPA) (Witte R P et al., 2004; J. Biomed. Mater. Res. 71A(3): 508-18), camphorquinone (CQ), 1-phenyl-1,2-propanedione (PPD) (Park Y J et al., 1999, Dent. Mater. 15(2): 120-7; Gamez E, et al., 2003, Cell Transplant. 12(5): 481-90), the organometallic complex Cp′Pt(CH(3))(3) (Cp′=eta(5)-C(5)H(4)CH(3)) (Jakubek V, and Lees AJ, 2004; Inorg. Chem. 43(22): 6869-71), 2-hydroxy-1-[4-(hydroxyethoxy)phenyl]-2-methyl-1-propanone (Irgacure 2959) (Williams C G, et al., 2005; Biomaterials. 26(11): 1211-8), dimethylaminoethyl methacrylate (DMAEMA) (Priyawan R, et al., 1997; J. Mater. Sci. Mater. Med. 8(7): 461-4), 2,2-dimethoxy-2-phenylacetophenone (Lee Y M et al., 1997; J. Mater. Sci. Mater. Med. 8(9): 537-41), benzophenone (BP) (Wang Y and Yang W. 2004; Langmuir. 20(15): 6225-31), flavin (Sun G, and Anderson VE. 2004; Electrophoresis, 25(7-8): 959-65).

The photoinitiation reaction can be performed using a variety of wave-lengths including UV (190-365 nm) wavelengths, and visible light (400-1100 nm) and at various light intensities (as described in Example 2 of the Examples section which follows). It will be appreciated that for ex vivo or in vivo applications, the photoinitiator and wavelengths used are preferably non-toxic and/or non-hazardous.

Cross-linking of the alginate constitute of the PEG-alginate polymer of the present invention is preferably performed in the presence of CaCl₂.

It will be appreciated that various concentrations of CaCl₂ can be used to polymerize the alginate constitute of the PEG-alginate polymer of the present invention. For example, as is shown in FIGS. 6a-b and Example 2 of the Examples section which follows, the present inventors used CaCl₂ at a concentration range between 5-20% in the preparation of the PEG-alginate polymers of the present invention.

Thus, the PEG-alginate polymer of the present invention (in which the PEG is interconnected to the alginate polymer network) can be prepared as follows. Briefly a precursor alginate solution (3.3. % w/v) is prepared by dissolving 3.3 gram of sodium alginate (Cat no. 71240, Fluka, Buchs, Switzerland) in 100 ml of de-ionized water and stirring over night. For the preparation of a PEG-alginate polymer, 4-kDa PEG-DA is added to the alginate precursor solution (3.3. % w/v) at a final concentration of 0.33% (w/v) of the 4-kDa PEG-DA and Igracure™2959 (a photoinitiator, Ciba Specialty Chemicals, Tarrytown, N.Y.) is added at a final concentration of 150 μg/ml. To obtain a homogenous solution, the PEG-alginate solution is centrifuged for 20 minutes at 3000 rcf and further de-gassed for 1 hour, following which the degassed solution (25 ml) is transferred to a square plastic Petri dish (120 mm×120 mm) and is allowed to dry for 2 days at room temperature on a perfectly level surface. Calcium cross-linking is accomplished by pouring 50 ml of a 15% w/v CaCl₂ solution directly onto the dehydrated alginate-containing dish. After a 15-minute incubation at room temperature in the presence of CaCl₂ (a cross-linker of the alginate component), the PEG constitute of the PEG-alginate solution is cross-linked in the presence of UV light (365 nm, 4-5 mW/cm²), following which the CaCl₂ solution is discarded and the film is gently peeled away from the dish and washed with de-ionized water. The PEG-alginate polymer film is further dried for 3-5 minutes under vacuum and 50° C. using a Gel Drying system (Hoefer Scientific Instruments).

As is mentioned before, in order to expose the substance included in the polymer film of the present invention to the lumen of the biological vessel of the present invention, the polymer film is rolled prior to its deployment inside the lumen of the biological vessel.

It will be appreciated that in order to access the lumen of small biological vessels such as blood vessels, urinary tract, digestive tract and the like, the rolled polymer film is preferably rolled over a small delivery vehicle capable of delivering and/or carrying the rolled polymer film into the lumen of the biological vessel. Such delivery vehicles can be, for example, an endoluminal stent, an endoluminal balloon catheter, and an endoluminal catheter.

Preferably, the polymer film of the present invention is rolled over a stent. The stent used by the present invention can be any stent known in the art, having any shape and/or dimensions [Lau, 2004 (Supra)] and made of any material and/or coating [e.g., a phosphorylcholine polymer (Lewis A L et al., Biomed Mater Eng. 2004; 14(4):355-70), a fluorinated polymer (Verweire I et al., J Mater Sci Mater Med. 2000 April; 11(4):207-12), degradable hyaluronan (Heublein B, et al., 2002; Int J Artif Organs. 25(12):1166-73)].

It will be appreciated that the stent used by the present invention can be a self-expandable stent that expands following its placement in the lumen of the blood vessel [e.g., Symbiot PTFE-covered stent (Burzotta F, et al., 2004; Chest. 126(2): 644-5) or RADIUS stent (Sunami K et al., 2003; J Invasive Cardiol. 15(1):46-8)] or a stent which is positioned over an angioplastic balloon, and which is expanded following the inflation of the balloon in the lumen of the blood vessel [e.g., a balloon expandable stent (Cohen D J., et al., 2004; Circulation. 110(5): 508-14)]. Preferably, the stent strut used by the present invention is positioned over an angioplastic balloon, i.e., a balloon catheter used for angioplasty.

Stents suitable for use along with the present invention can be purchased from any supplier of biomedical instruments such as Zoll Medical Corporation (Chelmsford, Mass., USA), Bioscorpio Investigational BioMedical & BioSurgical Products, (Belgium), Medtronic Inc. (Minneapolis, Minn., USA), Boston Scientific (Natik, Mass., USA), and Cordis Corporation (Miami, Fla., USA).

It will be appreciated that the polymer film rolled over the stent of the present invention can be placed into the biological vessel (e.g., blood vessel) using a catheter according to standard medical protocols (Leopold J A and Jacobs A K. 2001, Rev. Cardiovasc. Med. 2(4):181-9; Timmis A D. 1990; Br Heart J. 64(1): 32-5).

Once the rolled polymer film is inside the lumen of the biological vessel, the polymer film is preferably unrolled by expanding the stent towards the luminal wall of the biological vessel to thereby expose the luminal wall of the blood vessel to the substance included in or on the polymer film of the present invention.

It will be appreciated that the teachings of the present invention can be used during or following balloon angioplasty with or without stent deployment.

For example, balloon angioplasty with stent deployment can be performed using the rolled polymer film of the present invention (e.g., the PEG-alginate polymer). Such a polymer film is preferably coated with an antiproliferative agent (e.g., Paclitaxil) to prevent proliferation of smooth muscle cells, deposition of extracellular matrix and subsequently prevent restenosis.

Thus, according to another aspect of the present invention there is provided a method of preventing restenosis in an individual in need thereof.

The phrase “restenosis” refers to the process of re-narrowing the blood vessel following an angioplastic procedure such as balloon angioplasty and/or stent deployment.

As used herein, the term “individual” refers to any human being, male or female, at any age, which suffers from a disease, disorder or condition which is associated with narrowing of a blood vessel (i.e., stenosis). Non-limiting examples for such disease, disorder or condition include, atherosclerosis, diabetes, heart disease, vascular disease, peripheral vascular disease, coronary heart disease, unstable angina and non-Q-wave myocardial infarction, and Q-wave myocardial infarction.

The phrase “preventing” refers to inhibiting or arresting the development of restenosis. Those of skill in the art will be aware of various methodologies and assays which can be used to assess the development of restenosis, and similarly, various methodologies and assays which can be used to assess the reduction, remission or regression of restenosis.

The method is effected by inserting the rolled polymer film of the present invention (which includes the substance as described hereinabove) into the lumen of a blood vessel and unrolling such a polymer film in the lumen of the blood vessel to thereby expose the luminal wall of the blood vessel to the substance of the present invention and prevent restenosis in the individual.

It will be appreciated that the polymer film of the present invention can be coated or impregnated with a variety of drugs which promote endothelialization of the luminal wall of the blood vessel and thus promote vascular re-healing. Such drugs can be, for example, growth factors (e.g., VEGF, angiopeptin) and cytokines (e.g., M-CSF, IL-1beta, IL-8, beta-thromboglobulin, EMAP-II, G-CSF, IL-10) capable of promoting vascular re-healing.

Thus, according to yet another aspect of the present invention there is provided a method of promoting vascular re-healing in an individual in need of an angioplasty procedure.

As used herein, the phrase “angioplasty procedure” refers to inserting a catheter into a blood vessel, inserting a balloon using a catheter into a blood vessel, and/or inserting a stent strut positioned over a balloon into a blood vessel.

As is mentioned before, the polymer film of the present invention can be introduced into the blood vessel during an angioplasty procedure. It will be appreciated that such a polymer film can also prevent the adhesion of platelets associated with the angioplasty procedure by providing a thin, smooth barrier which protects the luminal wall from platelet activation and the subsequent thrombosis formation at the site of balloon inflation and/or stent deployment.

Thus, according to another aspect of the present invention there is provided a method of preventing thrombosis at a luminal wall of a blood vessel.

As used herein the phrase “thrombosis” refers to the formation, development, or presence of a thrombus (blood clot) in a blood vessel or the heart.

The method is effected by deploying the polymer film of the present invention in the luminal wall of the blood vessel as described hereinabove.

The polymer film of the present invention which is rolled over the stent as described above, is also suitable for the treatment of disorders associated with other biological vessels which require localized treatment for repairing or restoring function a vessel, cavity and/or lumen. Examples for such disorders include, but are not limited to, erosive esophagitis, esophageal laceration, esophageal ruptures and perforations, blockage of the renal arteries, ureters injuries, urethral injuries or stenosis, and renal vein thrombosis. Those of skills in the art are capable of selecting the appropriate substance which forms, coats or impregnates the polymer film of the present invention in each case, depending on the condition or disease to be treated.

For example, in order to treat erosive esophagitis, the polymer film of the present invention is preferably made from PEG-alginate at the approximate dimensions of 150 mm (length), 75 mm (width) and 200 μm (thickness) and includes proton pump inhibitors such as esomeprazole, omeprazole and lansoprazole (Raghunath A S et al., 2003, Clin. Ther. 25: 2088-101; Vakil N B et al., 2004, Clin. Gastroenterol. Hepatol. 2: 665-8).

Similarly, in order to treat blockage of the renal arteries, the polymer film of the present invention is preferably made from PEG-alginate at the approximate dimensions of 100-150 mm (length), 15-35 mm (width) and 200 μm (thickness) and includes anticoagulants such as clopidogrel, aspirin, and heparin.

In order to treat urethral injuries or stenosis, the polymer film of the present invention is preferably made from PEG-alginate at the approximate dimensions of 100-150 mm (length), 45-50 mm (width) and 200 μm (thickness) and may include an anti-hypotensive agent such as amezinium (Ishigooka M, et al., 1996; Int. Urogynecol. J. Pelvic. Floor Dysfunct. 7: 325-30).

It is expected that during the life of this patent many relevant polymer films will be developed and the scope of the term polymer film is intended to include all such new technologies a priori.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., Ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (Eds.) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., Ed. (1994); “Culture of Animal Cells—A Manual of Basic Technique” by Freshney, Wiley-Liss, N.Y. (1994), Third Edition; “Current Protocols in Immunology” Volumes I-III Coligan J. E., Ed. (1994); Stites et al. (Eds.), “Basic and Clinical Immunology” (8th Edition), Appleton & Lange, Norwalk, Conn. (1994); Mishell and Shiigi (Eds.), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., Ed. (1984); “Nucleic Acid Hybridization” Hames, B. D., and Higgins S. J., Eds. (1985); “Transcription and Translation” Hames, B. D., and Higgins S. J., Eds. (1984); “Animal Cell Culture” Freshney, R. I., Ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); “Absorbable and Biodegradable Polymers” Shalaby W. Shalaby, Karen J. L. Burg, Publisher: CRC Press, Boca Raton, Fla. (Oct. 27, 2003) ISBN: 0849314844; “Handbook of Biodegradable Polymers (Drug Targeting and Delivery)” A. J. Domb, Abraham J. Domb, Joseph Kost, David M. Wiseman, Publisher: T&F STM, London (Dec. 1, 1997) ISBN: 9057021536; “Synthetic Biodegradable Polymer Scaffolds (Tissue Engineering)” Anthony Atala, David J. Mooney, Publisher: Birkhauser Boston (Jan. 1, 1997) ISBN: 0817639195; all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Generation of a Balloon Catheter Rolled Over with a Drug-Eluting Sheet

In order to improve post-traumatic intravascular re-healing associated PCI, the present inventors have uncovered that a drug-eluting sheet can be applied on the internal margins of an endoluminal vascular injury using a balloon catheter rolled over with a drug-eluting sheet, as follows.

### Experimental Design

The biodegradable sheet—The biodegradable sheet (i.e., the polymer film of the present invention) can accommodate the site-specific release of both cytotherapeutic drugs and cellular factors according to the determined needs of the vascular repair process.

The biodegradable sheet can be prepared from a variety of materials such as biological materials and/or hybrid polymers (i.e., made of synthetic and biological materials), and can include anti-proliferative agents such as rapamycin, paclitaxel, tranilast, and trapidil, as well as factors which promote re-endothelialization such as Vascular Endothelial Growth Factor (VEGF), angiopeptin, and the like.

The sheet is designed to be biodegradable such that during the repair process, the material will eventually give way to subcellular tissue, with the non-toxic degradation products being released into the circulation and cleared from the body. The release of cytotherapeutic drugs, cellular factors, and degradation products are all controlled via the structural parameters of the preformed material, including chemical composition, polymeric chain length, cross-linking density, and hydrophobicity of the material.

The time period for degradation of the drug-eluting sheet can vary depending on the needs of the vascular repair process. Thus, degradation and drug delivery parameters can be designed for several days and up to several months.

Furthermore, the material is designed to be non-thrombogenic based on its anti-adhesive characteristics. The material does not necessarily support the adsorption of proteins and coagulations factors, including adhesion of platelets and circulation cells.

Examples for such materials include, but are not limited to, tissue plasminogen activator, reteplase, TNK-tPA, glycoprotein IIb/IIIa inhibitors (e.g., abciximab, eptifibatide, tirofiban), clopidogrel, aspirin, heparin and low molecular weight heparins such as enoxiparin and dalteparin (Reviewed in Buerke M and Rupprecht HJ, 2000. EXS 89:193-209).

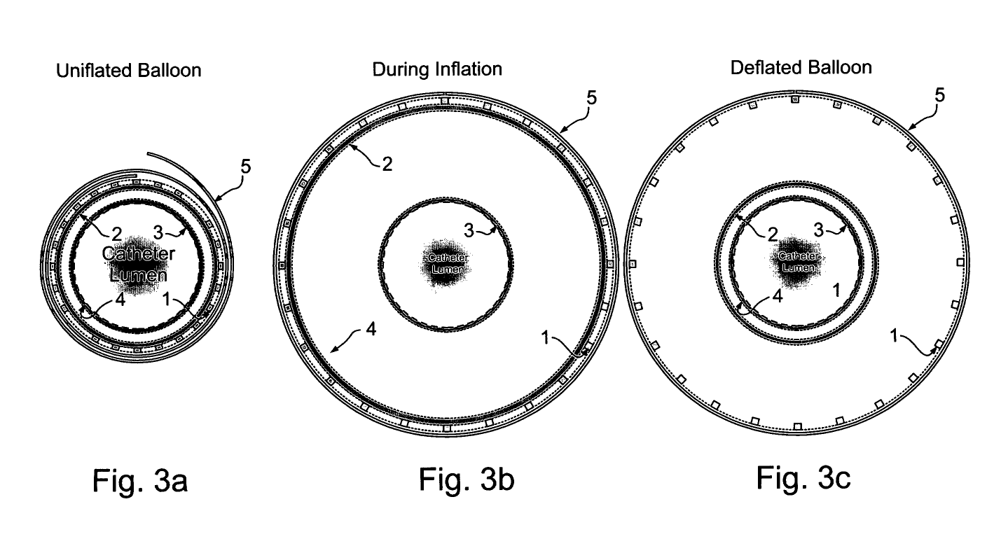

Modes of application of the drug-eluting sheet—As is shown in FIGS. 1-3, the biodegradable, drug-eluting sheet can be delivered onto the injury site of the vessel using an intravascular stent (FIGS. 1a-b). The polymer sheet is rolled over the stent and temporarily secured in place to allow for safe passage to the local target in the vasculature (FIG. 2). At the site of administration, the stent will be expanded with the rolled sheet overtop, causing the thin sheet to unroll and hug the internal margins of the target vessel. The biodegradable, drug-eluting sheet stays in place on the artery wall for the duration of its therapeutic function using the stent as an anchoring mechanism (FIGS. 3a-b).

The thin film is securely wrapped several times around a metallic stent and unravels onto the vessel wall during balloon inflation and stent deployment. After deployment, the metallic struts secure the film in place and ensure uniform material coverage of the vessel lumen. The non-thrombogenic film can be loaded with anti-proliferative drugs and growth factors for sustained, uniform release to the vessel wall.

### Example 2

### Endoluminal Hydrogel Films Made of Alginate and Polyethylene Glycol: Physical Characteristics

Materials and Experimental Methods

Materials—The following materials were purchased from the noted suppliers: Sodium Alginate (from brown algae; Fluka BioChemika, Buchs, Switzerland); Linear PEG-OH (4-kDa MW), triethylamine and sodium azide (Fluka; Buchs, Switzerland); Dichloromethane, Iodine, Sigmacotte®, and n-octanol (Sigma, St. Louis, Mo., USA; Aldrich, Sleeze, Germany; or Sigma-Aldrich, Steinheim, Germany); Acryloyl chloride and Toluene (Merck, Darmstadt, Germany); Calcium Chloride (Spectrum, NJ, U.S.A); phosphate buffered saline (D-PBS; Gibco, Scotland, UK); Diethyl ether (Bio Lab Ltd, Jerusalem, Israel); Igracure™2959 photoinitiator was generously donated by Ciba Specialty Chemicals (Tarrytown, N.Y.).

Synthesis of PEG Diacrylate—PEG-diacrylate (PEG-DA) was prepared from linear PEG, 4-kDa MW as described elsewhere (13, 19). Briefly, acrylation of PEG-OH was carried out under Argon by reacting a dichloromethane (DCM) solution of the PEG-OH with acryloyl chloride and triethylamine at a molar ratio of 1-OH to 1.5-acryloyl chloride to 1.5-triethylamine (0.2 gram PEG/ml DCM). The final product was precipitated in ice-cold diethyl ether and dried under vacuum overnight. The degree of the end-group conversion was tested using ¹H NMR and was found to be 97-99% (data not shown).

Preparation of ALG and PEG-ALG films: A precursor alginate solution (3.3% w/v) was prepared by dissolving 3.3 gram of sodium alginate in 100 ml of de-ionized water and stirred over night. PEG-ALG films were made with an alginate precursor solution containing 0.33% (w/v) of 4-kDa PEG-DA and 1.5 μl/ml of a photoinitiator stock solution (10 mg Igracure™2959 in 100 μl of 70% ethanol). The precursor solution was centrifuged for 20 minutes at 3000 rcf in 50 ml centrifuge tube (up to 30 ml in each tube). The solution was de-gassed for 1 hour and 25 ml were transferred into square plastic Petri dishes (120 mm×120 mm). The solution was dried at room temperature for 2 days on a perfectly level surface. Calcium cross-linking of the alginate films was accomplished by pouring 50 ml of CaCl₂ solution (15% w/v) directly into the dehydrated alginate-containing dish for 15 minutes incubation at room temperature. The PEG-containing films were cross-linked in the presence of UV light (365 nm, 4-5 mW/cm²). After cross-linking, the CaCl₂ solution was discarded and the film was gently peeled away from the dish and washed with de-ionized water before being dried for 3-5 minutes under vacuum and 50° C. using a Gel Drying system (Hoefer Scientific Instruments).

Preparation of PEG films—A PEG-DA precursor solution (16.5% w/v) was prepared by dissolving 0.91 gram of 4-kDa PEG-DA in 5.1 ml de-ionized water containing 410 μl of an Igracure™2959 stock. The solution was vortexed and centrifuged for 5 minutes at 3000 rcf. The PEG solution (3.4 ml) was then placed into a rectangular area (129 mm×87 mm) between two Sigmacotte®-treated glass plates separated by a 0.3 mm gap. The rectangular area is designated with an hydrophobic marker which delimits the PEG-DA solution into the rectangular to form a uniformly thick film. The PEG solution was cross-linked for 15 minutes in the presence of UV light (365 nm, 4-5 mW/cm²). After cross-linking, the PEG film was gently peeled away from the glass plates and dried under vacuum for 60 minutes with mild heating using a Gel Drying system.

Swelling Properties—Dehydrated films were cut into 11.7-mm or 10.1-mm diameter discs using a stainless-steel punch. The thickness, radius, and weight of the films were measured and logged prior to and after incubation in de-ionized water containing 0.1% sodium azide. The weight swelling ratio (SR_{w}) was calculated by dividing the weight of the swollen film by the weight of the dry film. The radial and thickness swelling ratios (SRr and SRt, respectively) were similarly calculated.

Mechanical Properties—The uniaxial mechanical properties of the hydrated and dehydrated ALG and PEG-ALG polymer films (with and without UV photoinitiation) were evaluated using an Instron™ 5544 single column material testing system with Merlin software. The stress-strain characteristics of 10-mm-wide dumbbell strips of polymer film cut from sheets of cross-linked PEG or PEG-ALG (100-mm long) were measured by constant straining (0.1 mm/sec) between two rigid grasps. The films were strained to failure and the force-displacement is recorded. The Merlin software automatically converts the raw data into a stress-strain relationship describing the material properties of each sample. The maximum tensile strength of the polymer films was presented as the ultimate stress and the elastic modulus was the average slope of the lower portion of the stress-strain curve (between 5-15% strain).

Degradation—The degradation of alginate-based films was assessed by measuring the modulus of the film after incubation in different ionic concentrations of saline solution (D-PBS). Dumbbell strips of ALG and PEG-ALG polymer films (10-mm-wide) were incubated in D-PBS (15, 37, 75, and 150 mM) for up to one week; each strip was placed into 30 ml of the saline solution and incubated at 37° C. with constant shaking. The strips were removed from the saline solution at certain time intervals and the mechanical properties of the strip were measured as before. In some experiments the saline was replenished between each time interval while in other experiments the same saline was used throughout.

Experimental Results

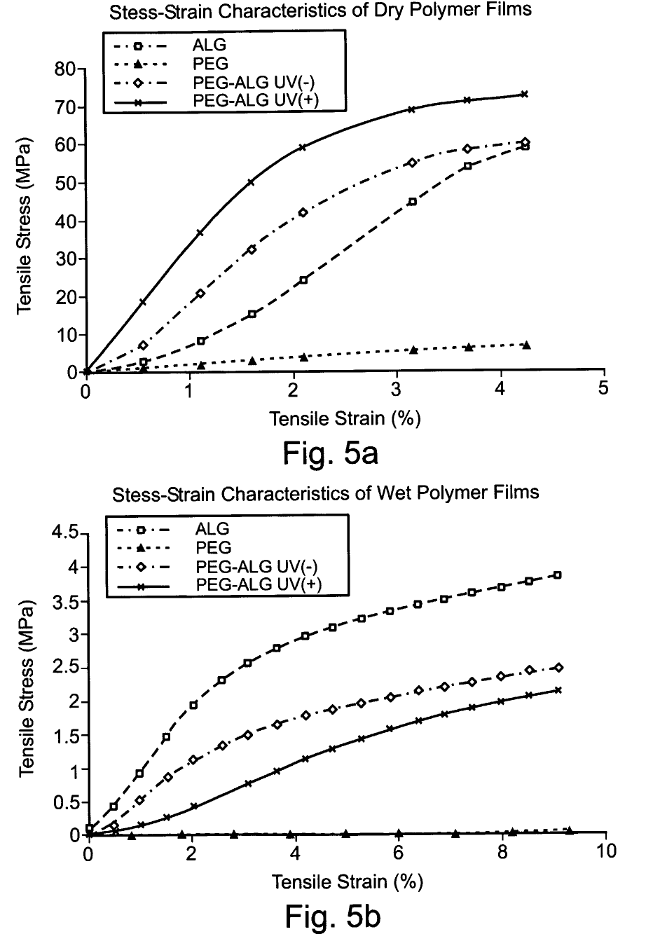

The alginate component is dominant in the ALG-PEG polymer film—Polymer films were made from alginate or PEG, or a composite of the two. The films were dehydrated and cross-linked in preparation for mechanical properties testing. The stress-strain characteristics of the films were recorded and are summarized in FIGS. 5a-b and Table 1, hereinbelow. The uniaxial stress-strain characteristics were found to be non-linear and highly influenced by the hydration of the polymer films; thus, the properties of the dehydrated polymer films were approximately an order of magnitude higher than the hydrated films [n=6, p<0.01; compare FIG. 5a (Dry) with FIG. 5b (Wet)]. On the other hand, pure alginate films were found to be much stronger than pure PEG films regardless of their hydration state (FIGS. 5a-b). Thus, the mechanical properties data evinces that the alginate is the dominant structural component in the composite network. Furthermore, the addition of PEG to the alginate films did not significantly improve their mechanical properties (n=5, p>0.05; FIGS. 5a-b). Interestingly, the maximum tensile strength (ultimate stress) of the dry polymer films made with pure alginate was not statistically different from that of films made from the PEG-alginate precursors. However, upon hydration, the PEG-ALG films become significantly weaker (n=5, p<0.01). Moreover, the free-radical polymerization of the PEG-DA precursors by exposure to UV light did not significantly alter the ultimate stress or modulus of the PEG-ALG films (n=6, p>0.05).

TABLE 1
Materials properties of wet and dry polymer films

| | | | |
|---|---|---|---|
| ALG | PEG | ALG-PEG UV (−) | ALG-PEG UV (+) |

Wet Polymer films

| | | | | |
|---|---|---|---|---|
| Ultimate Stress | 9.7 ± 1.11 | — | 7.7 ± 0.88 | 6.4 ± 0.76 |
| (MPa) | | | | |
| Modulus (MPa) | 0.201 ± 0.02 | 0.002 ± 1 × 10 ⁵ | 0.131 ± 0.01 | 0.147 ± 0.01 |

Dry Polymer films

| | | | | |
|---|---|---|---|---|
| Ultimate Stress | 50.5 ± 3.4 | 9.8 ± 0.3 | 48.3 ± 4.1 | 57.6 ± 5.8 |
| (MPa) | | | | |
| Modulus (MPa) | 24.8 ± 2.85 | 2.18 ± 0.06 | 22.0 ± 1.31 | 28.1 ± 2.47 |

Table 1: The ultimate stress and modulus (expressed in MPa) of the wet and dry polymer films of the present invention are presented. ALG = Alginate; PEG = polyethylene glycol; ALG-PEG UV (−) = PEG-alginate films in the absence of free-radical polymerization; ALG-PEG UV (+) = PEG-alginate films following free-radical polymerization.

Swelling properties reveal dominant effect of the alginate network—The swelling properties of the PEG-ALG films were assessed by measuring the thickness, diameter, and weight of dehydrated disks prior to or following hydration. A summary of the swelling characteristics is detailed in Table 2, hereinbelow. As is shown in Table 2, hereinbelow, the high swelling ratios of the PEG films demonstrate that these films absorb significantly more water than their alginate counterparts. In contrast, the swelling ratios of the alginate films were minimal, particularly the radial swelling ratio, which is effectively unchanged during hydration (n=6, p<0.01). In addition, the composite PEG-ALG films exhibited swelling characteristics which are identical to the ALG films, demonstrating the dominant influence of the alginate network. It is worth mentioning that exposure of composite PEG-ALG films to polymerization by UV light did not significantly alter their swelling properties (n=6, p>0.05).

TABLE 2
Swelling properties of polymer films

| Swelling Ratio | ALG | PEG | PEG-ALG UV (−) | PEG-ALG UV (+) |
|---|---|---|---|---|
| Thickness | 1.38 ± 0.165 | 5.86 ± 0.883 | 1.39 ± 0.168 | 1.40 ± 0.252 |
| Radial | 1.03 ± 0.008 | 1.37 ± 0.041 | 1.02 ± 0.007 | 1.04 ± 0.011 |
| Weight | 1.50 ± 0.129 | 13.8 ± 0.486 | 1.46 ± 0.186 | 1.69 ± 0.175 |

Table 2: The swelling properties of the polymer films of the present invention are presented. ALG = Alginate; PEG = polyethylene glycol; ALG-PEG UV (−) = PEG-alginate film in the absence of free-radical polymerization; ALG-PEG UV (+) = PEG-alginate film following free-radical polymerization.

The concentration of the CaCl₂ cross-linker affects the swelling and integrity of the alginate network—The effect of CaCl₂ cross-link concentration on the integrity of the alginate films was assessed by measuring the swelling ratio following cross-linking. Evidently, as indicated in FIGS. 6a-b, the calcium levels used to cross-link the films after dehydration exhibited a marked impact on hydration properties. The distribution of the swelling ratio versus CaCl₂ concentration indicates an optimal concentration of 15% for minimal swelling. Over-saturation of the cross-linking solution resulted in poor alginate cohesion and substantially higher swelling characteristics. Alternatively, insufficient amounts of the cross-linker reduced the integrity of the alginate network and resulted in slightly increased swelling during hydration (n=9, p<0.05). The relationship between CaCl₂ concentration and swelling characteristics for ALG and PEG-ALG films was statistically indistinguishable (n=9, p>0.05).

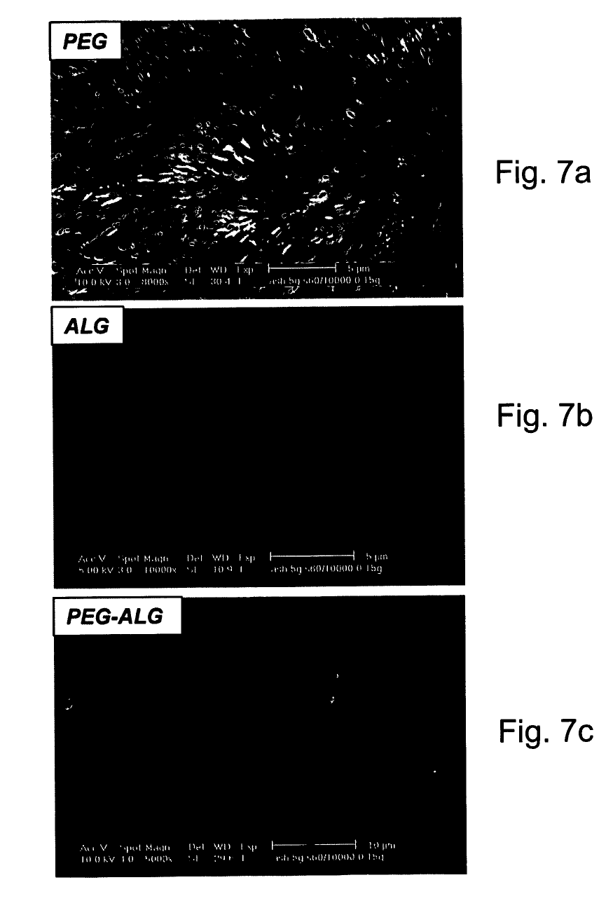

Scanning electron microscopy revealed topographic characteristics of the PEG-ALG films—Scanning electron micrographs of cross-linked PEG, alginate, and PEG-ALG films revealed the topographic characteristics of each material (FIGS. 7a-c). As is shown in FIG. 7a, the highly hydrophilic PEG films formed large pores (>100 nm) upon dehydration and exhibit non-uniform topography. In contrast, the alginate films were densely packed and highly homogeneous as indicated by the absence of micro-porous structures and relatively smooth surface (FIG. 7b). On the other hand, as is shown in FIG. 7c, the combination of PEG to the alginate films only slightly modified the surface topography in that the PEG-ALG films exhibited a characteristically rough surface with micron-scale pits and mounds (˜1 μm diameter).

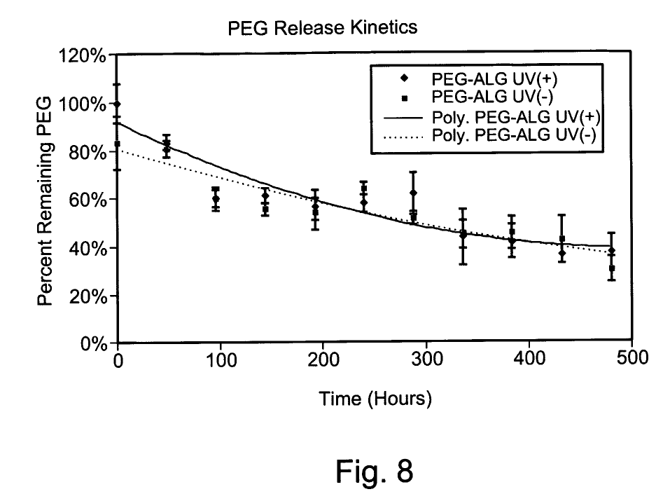

Kinetics of PEG release reveals a significant decrease in the PEG component in the presence of PBS—The release of PEG from the composite PEG-ALG films was assessed during a three-week incubation period in the presence of PBS by measuring the quantities of entrapped PEG in the films using iodoacetate. FIG. 8 depicts the fraction of remaining PEG in the films (relative to the initial quantities of PEG) as a function of time. As is shown in FIG. 8, no significant difference in the PEG release profile was observed between UV-treated (UV+) and untreated (UV−) PEG-ALG composite films (n=10, p<0.01). In addition, these quantification data demonstrate that following three weeks of incubation less than 40% of the PEG is present in the films. It will be appreciate that since the quantification assay necessitates 90-min incubation in dilute iodoacetate solution prior to measurement, some of the initially unbound PEG at time-zero is likely washed out, thus altering the release profile of PEG.

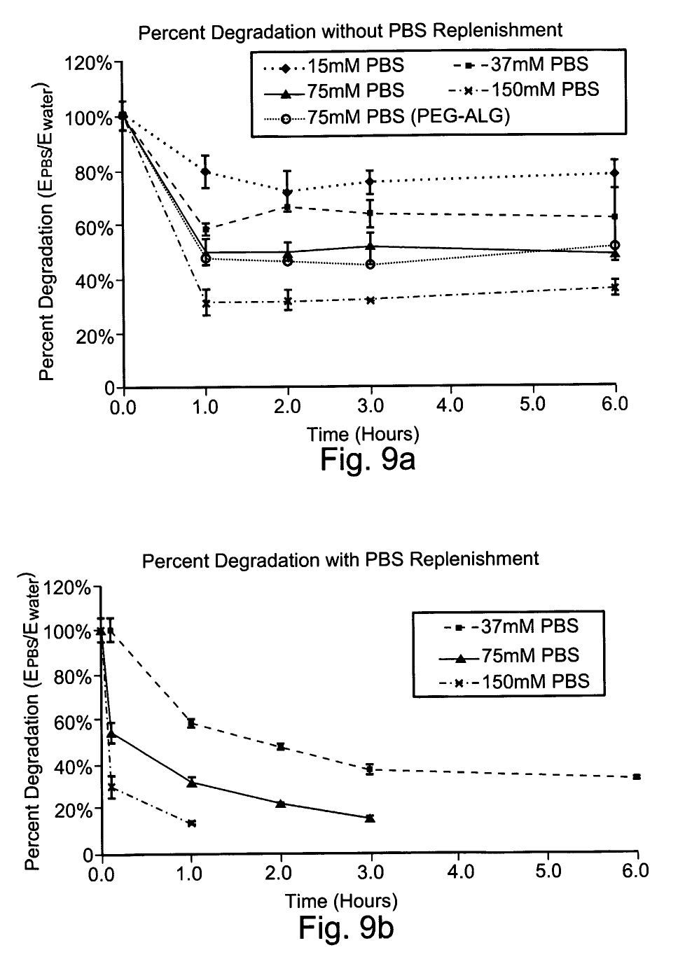

The PEG-ALG and the ALG films of the present invention maintain stable material modulus following the initial degradation in the presence of phosphate buffer saline (PBS)—The degradation properties of the alginate and composite PEG-ALG films were assessed by measuring the material modulus of the film before and after incubation in water or PBS. The degradation of the alginate network in various concentrations of PBS is summarized in FIG. 9a. While in the presence of water, the alginate films maintain their stability for several months without a significant decrease in material modulus (data not shown), in the presence of PBS, the alginate films exhibited a significant reduction in the film stability. As is shown in FIG. 9a, almost immediately after incubation with 150 mM PBS, a significant reduction in the stability of the alginate network was observed. After the initial deterioration in modulus, the films reached a new steady-state modulus without any further degradation observed (up to one week). For any given concentration of PBS, the alginate films demonstrated a proportionate and immediate reduction in their modulus without further degradation. Similarly, the PEG-ALG films exhibited identical degradation characteristics (FIG. 9a). Further analysis of the film modulus following replenishment of the PBS buffer at each measurement time interval revealed that the degradation characteristics of the alginate films were affected primarily by the ionic strength of the buffer solution and the replenishment intervals (FIG. 9b). At high concentrations of replenished PBS, the rapid deterioration of the alginate network resulted in an inability to continue the modulus measurements beyond a few measurement intervals.

Altogether, these results demonstrate that the combination of alginate and PEG provides excellent compliance and physical strength to endure the physical demands of the hemodynamic environment and to be held affixed to the vessel lumen using the stent struts.

### Example 3

### Endoluminal Hydrogel Films Made of Alginate and Polyethylene Glycol: Drug-Eluting Properties and Feasibility of Polymer Depolyment

Materials and Experimental Methods

Materials—were purchased from the suppliers detailed in Example 2 hereinabove. Paclitaxel (Medixel 30 mg/5 ml) was purchased from TARO Pharmaceutical Ltd., Haifa Bay, Israel.

Preparation of ALG and PEG-ALG films: A precursor alginate solution (3.3% w/v) was prepared by dissolving 3.3 gram of sodium alginate in 100 ml of de-ionized water and stirred over night. PEG-ALG films were made with an alginate precursor solution containing 0.33% (w/v) of 4-kDa PEG-DA and 1.5 μl/ml of a photoinitiator stock solution (10 mg Igracure™ 2959 in 100 μl of 70% ethanol). The precursor solution was mixed directly with commercially available Paclitaxel suspension (Medixel 30 mg/5 ml, TARO Pharmaceutical LTD., Haifa, Israel) and then centrifuged for 20 minutes at 3000 rcf in 50 ml centrifuge tube (up to 30 ml in each tube). The solution was de-gassed for 1 hour and 25 ml were transferred into square plastic Petri dishes (120 mm×120 mm). The solution was dried at room temperature for 2 days on a perfectly level surface. Calcium cross-linking of the alginate films was accomplished by pouring 50 ml of CaCl₂ solution (15% w/v) directly into the dehydrated alginate-containing dish for 15 minutes incubation at room temperature. The PEG-containing films were cross-linked in the presence of UV light (365 nm, 4-5 mW/cm²). After cross-linking, the CaCl₂ solution was discarded and the film was gently peeled away from the dish and washed with de-ionized water before being dried for 3-5 minutes under vacuum and 50° C. using a Gel Drying system (Hoefer Scientific Instruments).

Paclitaxel release—Small samples (circular discs, 8 mm) of the Paclitaxel films were placed in a solution of octanol and phosphate buffered saline (PBS) or water at a proportion of 5 ml Octanol and 10 ml PBS (or water). The solution, including the film disc, was shaken continuously at 37° C. for several days. The amount of Paclitaxel in the octanol phase of the solution was measured using a spectrophotometer at 232 nm. Measurements were carried out periodically and the amount of drug released was normalized to baseline values for control films containing no drug. The protocol for drug release experiment is documented in previous studies by Jackson et al (Jackson J K, et al, 2002, Pharmaceutical Research 19(4):411-417).

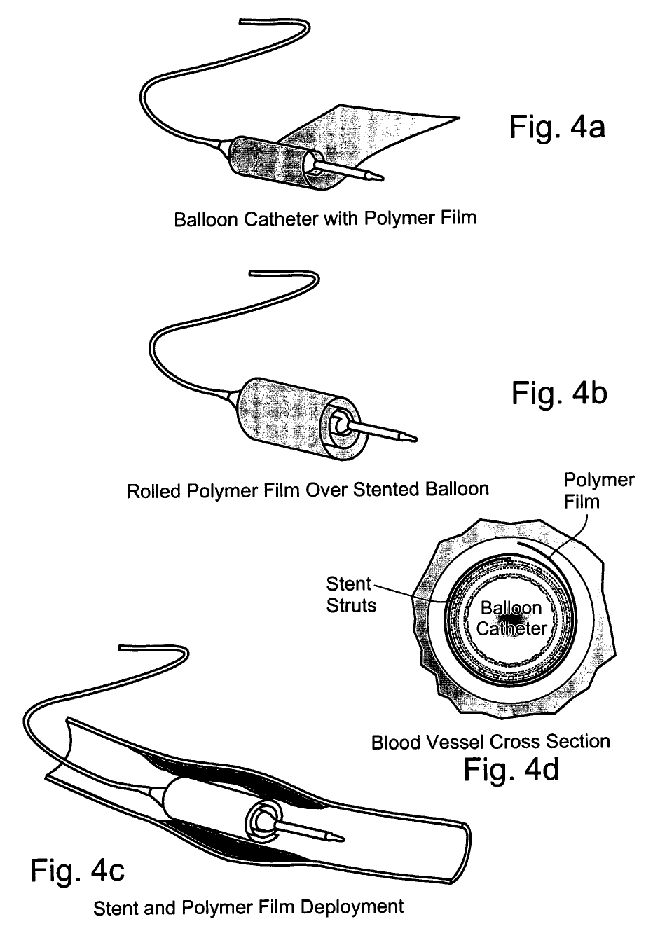

Film deployment—Endoluminal deployment of the PEG-ALG films was tested in rabbit abdominal aortic tissue samples using an in vitro organ culture system. The films (50-100 μm thick) were cut to appropriate dimensions and wrapped around an ACS RX MultiLink coronary stent (diameter 3.5 mm, stent length 15 mm) requiring an expansion pressure of 6 atm. and having a burst pressure of 8 atm. (Advanced Cardiovascular Systems, Inc., Temecula, Calif., USA). Wrapping the film around the stent was accomplished by placing the pre-wetted film over the stent, wrapping it around for several times, and securing in place with a thin line of Bio-Glue (BG3002-5-G, Cryolife Inc. Marietta, Ga., USA) on the periphery of the film (as illustrated in FIGS. 4a-d). The films were inserted through the organ culture system into the lumen of the aorta tissue sample. Inflation of the balloon caused the film to unravel onto the endolumenal surface as illustrated in FIGS. 3a-c. Fluid was circulated in the artery lumen to ensure adequate adherence of the film under shear conditions (up to 100 dynes/cm² at the lumen interface).

Experimental Results

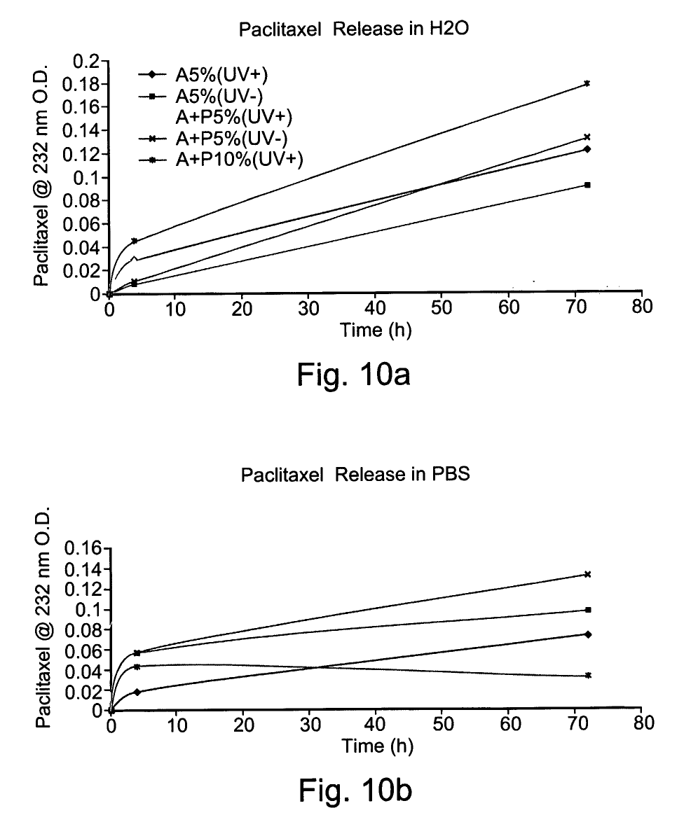

Paclitaxel release—The release of the paclitaxel drug was recorded at time zero and after 4 and 72 hours under continuous shaking with constant temperature of 37° C. As is shown in FIG. 10, the profile of drug release in PBS was significantly faster than in water. Such differences are likely attributed to the different ionic strengths of the buffer in which the films are placed.

Film deployment—The feasibility of inserting an endoluminal polymer film using a balloon catheter and a stent according to the method of the present invention was tested in the ex vivo flow circuit. The stent and endoluminal film were successfully deployed and endured the flow of fluid through the artery lumen. The system was allowed to operate for 24 hours under steady-state flow conditions. At the end of the experiment, the film was checked visually to ensure adherence to the artery wall. The stent struts were visually inspected to ensure that they tightly affix the film onto the vessel wall as illustrated in FIGS. 3a-c. The deployment study demonstrated feasibility of application using wrapped around endoluminal films.

### General Analysis and Discussion of Examples 1-3

The present study describes the development of PEG-alginate hydrogel films and characterizes their physiochemical properties. The films are created using a cross-linking scheme designed to significantly increase the strength of the load bearing alginate network. The uniaxial tensile testing demonstrated that the compliance of the hydrogel films is enhanced using an interpenetrating network of PEG in the alginate hydrogel. The present study demonstrates the degradability of the PEG-alginate films as a function of ionic concentration of buffer solution; the anisotropic swelling of the films which makes them suitable for endoluminal applications; and the drug release properties of the PEG-alginate films which are characterized using the anti-proliferative agent called Paclitaxel. Finally, the deployment of the PEG-alginate films is demonstrated ex vivo using a circulating organ culture system with rabbit aortas.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1-133. (canceled)

134. A polymer film comprising crosslinked polyethylene glycol (PEG) and a biologically derived polymer.

135. The polymer film of claim 134, wherein said biologically derived polymer comprises a material selected from the group consisting of ionically crosslinked polymers, fibrinogen, collagen, albumin, fibrin, gelatin and bacterial cellulose.

136. The polymer film of claim 135, wherein said ionically crosslinked polymer is selected from the group consisting of an alginate, hyaluronic acid and alginate-fibrin.

137. The polymer film of claim 134, wherein said biologically derived polymer comprises an alginate.

138. The polymer film of claim 134, being substantially biodegradable.

139. The polymer film of claim 134, further comprising at least one drug.

140. The polymer film of claim 139, wherein said drug is selected from the group consisting of an anti-adhesive substance, an anti-thromobogenic substance, an antiproliferative drug, a growth factor, a cytokine and an immunosuppressant drug.

141. A medical device, comprising a polymer film of claim 134.

142. The medical device of claim 141, configured for the delivery of a drug.

143. A polymer film comprising crosslinked polyethylene glycol (PEG) and at least one drug.

144. The polymer film of claim 143, wherein said at least one drug is selected from the group consisting of an anti-adhesive substance, an anti-thromobogenic substance, an antiproliferative drug, a growth factor, a cytokine and an immunosuppressant drug.

145. The polymer film of claim 143, further comprising a biologically derived polymer.

146. The polymer film of claim 143, being substantially biodegradable.

147. A medical device, comprising a polymer film of claim 143.

148. The medical device of claim 147, configured for the delivery of a drug.

149. A method of exposing a luminal wall of a biological vessel to a substance, comprising:
(a) inserting a rolled polymer film including the substance into a lumen of the biological vessel; and
(b) unrolling said rolled polymer film in the lumen of the biological vessel thereby exposing the luminal wall of the biological vessel to the substance
wherein said polymer film comprises cross-linked polyethylene glycol (PEG).

150. The method of claim 149, wherein said rolled polymer film is rolled over a stent.

151. The method of claim 150, wherein said inserting said rolled polymer is effected using a catheter.

152. The method of claim 150, wherein said unrolling said rolled polymer is effected using a self-expandable stent.

153. The method of claim 149, wherein said polymer film is biodegradable.

154. The method of claim 149, wherein said polymer film further comprises a biologically derived polymer.

155. The method of claim 154, wherein said biologically derived polymer comprises a material selected from the group consisting of ionically crosslinked polymers, fibrinogen, collagen, albumin, fibrin, gelatin and bacterial cellulose.

156. The method of claim 155, wherein said ionically crosslinked polymer is selected from the group consisting of an alginate, hyaluronic acid and alginate-fibrin.

157. The method of claim 154, wherein said biologically derived polymer comprises an alginate.

158. The method of claim 149, said polymer film further comprising a drug.

159. The method of claim 158, wherein said drug is selected from the group consisting of an anti-adhesive substance, an anti-thromobogenic substance, an antiproliferative drug, a growth factor, a cytokine and an immunosuppressant drug.

160. The method of claim 149, wherein said biological vessel is selected from the group consisting of a blood vessel, an artery, a vein, an air tract vessel, a urinary tract vessel, and a digestive tract vessel.

161. The method of claim 149, wherein said biological vessel is a blood vessel and said exposing substantially prevents restenosis in said blood vessel.

162. The method of claim 149, wherein said biological vessel is a blood vessel, wherein said substance is capable of promoting vascular re-healing and said exposing substantially promotes vascular re-healing in said blood vessel.

163. A medical device comprising, an expandable stent covered by a polymer film including cross-linked polyethylene glycol (PEG).

164. The medical device of claim 163, wherein said expandable stent is a self-expanding stent.

165. The medical device of claim 163, wherein said expandable stent is a balloon expandable stent.

166. The medical device of claim 163, wherein said polymer film further comprises a biologically derived polymer.

167. The medical device of claim 166, wherein said biologically derived polymer comprises a material selected from the group consisting of ionically crosslinked polymers, fibrinogen, collagen, albumin, fibrin, gelatin and bacterial cellulose.

168. The medical device of claim 167, wherein said ionically crosslinked polymer is selected from the group consisting of an alginate, hyaluronic acid and alginate-fibrin.

169. The medical device of claim 166, wherein said biologically derived polymer comprises a cross-linked alginate.

170. The medical device of claim 163, said polymer film further comprising a drug.

171. The medical device of claim 170, wherein said drug is selected from the group consisting of an anti-adhesive substance, an anti-thromobogenic substance, an antiproliferative drug, a growth factor, a cytokine and an immunosuppressant drug.

172. A method of preparing a polymer film, comprising:
a) combining a polyethylene glycol (PEG) and a second, ionically polymerizable, substance to yield a mixture;
b) forming a film of said mixture;
c) initiating polymerization of said PEG; and
d) initiating ionic polymerization of said second substance
thereby preparing the polymer film.

173. The method of claim 172, wherein said second substance is selected from the group consisting of alginate, hyaluronic acid and alginate-fibrin.

174. The method of claim 172, wherein said polymerization of said PEG is light initiated free-radical polymerization.

175. The method of claim 172, further comprising adding a drug to said mixture.

176. A method of preparing a polymer film, comprising:
a) combining a polyethylene glycol (PEG) and a drug to yield a mixture;
b) forming a film of said mixture; and
c) initiating polymerization of said PEG.
thereby preparing the polymer film.

177. The method of claim 176, wherein said polymerization of said PEG is light initiated free-radical polymerization.
